SEQUENCE LISTING (14 sequences)

SEQ ID NO: 1 - DNA, 1595 bp, Homo Sapiens
ccatcctaatacgactcactatagggctcgagcggccgcccgggcaggttaactgtccaccagaaaggactgctctttgggtgagttgaacttcttccattatagaaagaattgaaggctgagaaactcagcctctatcatgtggaacagctctgacgccaacttctcctgctaccatgagtctgtgctgggctatcgttatgttgcagttagctggggggtggtggtggctgtgacaggcaccgtgggcaatgtgctcaccctactggccttggccatccagcccaagctccgtacccgattcaacctgctcatagccaacctcacactggctgatctcctctactgcacgctccttcagcccttctctgtggacacctacctccacctgcactggcgcaccggtgccaccttctgcagggtatttgggctcctcctttttgcctccaattctgtctccatcctgaccctctgcctcatcgcactgggacgctacctcctcattgcccaccctaagctttttccccaagttttcagtgccaaggggatagtgctggcactggtgagcacctgggttgtgggcgtggccagctttgctcccctctggcctatttatatcctggtacctgtagtctgcacctgcagctttgaccgcatccgaggccggccttacaccaccatcctcatgggcatctactttgtgcttgggctcagcagtgttggcatcttctattgcctcatccaccgccaggtcaaacgagcagcacaggcactggaccaatacaagttgcgacaggcaagcatccactccaaccatgtggccaggactgatgaggccatgcctggtcgtttccaggagctggacagcaggttagcatcaggaggacccagtgaggggatttcatctgagccagtcagtgctgccaccacccagaccctggaaggggactcatcagaagtgggagaccagatcaacagcaagagagctaagcagatggcagagaaaagccctccagaagcatctgccaaagcccagccaattaaaggagccagaagagctccggattcttcatcggaatttgggaaggtgactcgaatgtgttttgctgtgttcctctgctttgccctgagctacatccccttcttgctgctcaacattctggatgccagagtccaggctccccgggtggtccacatgcttgctgccaacctcacctggctcaatggttgcatcaaccctgtgctctatgcagccatgaaccgccaattccgccaagcatatggctccattttaaaaagagggccccggagtttccataggctccattagaactgtgaccctagtcaccagaattcaggactgtctcctccaggaccaaagtggccaggtaataggagaataggtgaaataacacatgtgggcattttcacaacaatctctccccagcctcccaaatcaagtctctccatcacttgatcaatgtttcagccctagactgcccaaggagtattattaattattaataaatgaattctgtgcttttaaaaaaaaaaaaataaaaaaagaaaaaaaaaaaaaaaaaaaaaaaaaaaaa

SEQ ID NO: 2 - protein, 396 aa, Homo Sapiens
MWNSSDANFSCYHESVLGYRYVAVSWGVVVAVTGTVGNVLTLLALAIQPKLRTRFNLLIANLTLADLLYCTLLQPFSVDTYLHLHWRTGATFCRVFGLLLFASNSVSILTLCLIALGRYLLIAHPKLFPQVFSAKGIVLALVSTWVVGVASFAPLWPIYILVPVVCTCSFDRIRGRPYTTILMGIYFVLGLSSVGIFYCLIHRQVKRAAQALDQYKLRQASIHSNHVARTDEAMPGRFQELDSRLASGGPSEGISSEPVSAATTQTLEGDSSEVGDQINSKRAKQMAEKSPPEASAKAQPIKGARRAPDSSSEFGKVTRMCFAVFLCFALSYIPFLLLNILDARVQAPRVVHMLAANLTWLNGCINPVLYAAMNRQFRQAYGSILKRGPRSFHRLH

SEQ ID NO: 3 - DNA, 23 bp, Homo Sapiens
  modified_base 6, 12, 15, 21: n is inosine
gaymgntayytngcnathgtnca

SEQ ID NO: 4 - DNA, 23 bp, Homo Sapiens
  modified_base 6, 12, 15: n is inosine
gaymgntayytngcnathgtcca

SEQ ID NO: 5 - DNA, 23 bp, Homo Sapiens
  modified_base 6, 9, 11, 12, 18, 20: n is inosine
rmrtanadnannggrttnanrca

SEQ ID NO: 6 - DNA, 23 bp, Homo Sapiens
  modified_base 6, 9, 11, 12, 18: n is inosine
rmrtanadnannggrttnacrca

SEQ ID NO: 7 - DNA, 24 bp, Homo Sapiens
gcaaagcagaggaacacagcaaac

SEQ ID NO: 8 - DNA, 22 bp, Homo Sapiens
gccaaagcccagccaattaaag

SEQ ID NO: 9 - DNA, 33 bp, Homo Sapiens
ctcagaattcatcatgtggaacagctctgacgc

SEQ ID NO: 10 - DNA, 31 bp, Homo Sapiens
tggtgtctagagtcacagttctaatggagcc

SEQ ID NO: 11 - DNA, 20 bp, Homo Sapiens
gactcactatagggctcgag

SEQ ID NO: 12 - DNA, 22 bp, Homo Sapiens
attgatcaagtgatggagagac

SEQ ID NO: 13 - DNA, 19 bp, Homo Sapiens
cacactggctgatctcctc

SEQ ID NO: 14 - DNA, 19 bp, Homo Sapiens
gggatgtagctcagggcaa

(19) United States
(12) Patent Application Publication
(10) Pub. No.: US 2002/0187947 A1
(43) Pub. Date: Dec. 12, 2002

(54) Inflammation-related gene

(76) Inventors: Gabor Jarai, Horsham (GB); Paul Roy Cooper, Birmingham (GB); Shida Yousefi, Bern (CH)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 09/798,710

(22) Filed: Mar. 2, 2001

(51) Int. Cl. A61K 48/00; C12Q 1/68; G01N 33/53; A61K 38/17

(52) U.S. Cl. 514/44; 514/12; 435/7.1; 435/6

(57) ABSTRACT

The use of (A) a polypeptide comprising the amino acid sequence of SEQ ID NO:2, or a functionally equivalent variant of that amino acid sequence, or (B) a polynucleotide comprising a nucleotide sequence encoding the polypeptide (A), or (C) an antibody which is immunoreactive with the polypeptide (A), or (D) an antisense oligonucleotide comprising a nucleotide sequence complementary to that of polynucleotide (B), or (E) a polynucleotide probe comprising at least 15 consecutive nucleotides of (B), in a pharmaceutical for the diagnosis or treatment of a neutrophil-associated inflammatory disease.

This application claims benefit of U.S. Provisional Application No. ______, filed Jan. 4, 2001, which was converted from U.S. application Ser. No. 09/518,832, and which is incorporated by reference.

The present invention relates to the use of a disease-related related G-protein coupled receptor gene, designated EX33, the protein molecule encoded by EX33, and related molecules in diagnostic and therapeutic applications, and to the use of the protein encoded by EX33 as a therapeutic target.

Cells that are attracted into tissues during inflammation include various leukocytes, particularly inflammatory phagocytes such as neutrophilic and eosinophilic granulocytes and monocytes. Neutrophils have been associated with inflammation and tissue destruction in neutrophil associated respiratory diseases such as chronic obstructive pulmonary disease (COPD), including chronic bronchitis and emphysema associated therewith, and adult respiratory distress syndrome (ARDS), inflammatory bowel diseases such as Crohn's disease and ulcerative colitis, and rheumatoid arthritis.

Critical steps in the action of leukocytes in inflammatory conditions include the migration of these cells into the tissues, e.g. into the airways in respiratory inflammations or to the joints in rheumatoid arthritis, cell activation and the release of a range of inflammatory mediators, leukotrienes, oxygen radicals, proteases. Signals that are needed for leukocyte migration and activation are often communicated through receptors that belong to the seven transmembrane-spanning G-protein coupled receptor (GPCR) superfamily. The GPCR gene family is the largest known receptor family. GPCRs are transducers of extracellular signals and they allow tissues to respond to a wide array of signalling molecules.

For example, leukocyte migration involves the arrest and firm adhesion of blood cells on endothelial surfaces and the migration through the endothelium into the interstitium and from there to particular microenviroments. The movement of granulocytes (and other leukocytes) into tissues is regulated by chemotactic factors (chemokines) that are of fundamental importance for each of these steps. The chemokines, displayed on the surface of endothelial cells, interact with their cognate receptors on granulocytes and other leukocytes, and firm adhesion is then followed by transmigration through tissues to the target microenviroment. The cognate receptors of chemokines are related members of a sub-family of GPCRs. In addition to chemotaxis, members of the GPCR super-family are also involved in a number of other signalling events that can lead to the activation of various cellular processes such as those observed in inflammatory conditions for example through coupling to intracellular calcium release or cAMP generation.

Amino acid sequence comparison between the different GPCRs revealed that sequence similarity can vary over a broad range and different sub-families of GPCRs often share no significant sequence similarity. However, all these receptors have in common seven transmembrane helices (TM-I through -VII) connected by three intracellular and three extracellular loops.

Various cloning strategies and database mining approaches led to the cloning of a number of GPCRs that are characterised by that common structure. The identification of ligands, however, lags behind and there are a large number of GPCR genes whose protein products, using sequence similarity and predicted 3D structure as the criteria, are members of the GPCR family, but for which the ligands are not known. These receptors are commonly known as orphan G-protein coupled receptors (oGPCRs).

G-protein coupled receptors are important targets in therapeutic applications because they are involved in a wide variety of physiological and pathological processes. It is estimated that 60-70% of currently marketed drugs indeed act on members of the GPCR superfamily.

One method, which can be used to identify GPCRs is degenerate primer polymerase chain reaction (PCR). In this method two mixtures of oligonucleotides corresponding to two selected, conserved regions of a GPCR gene sub-family are synthetised and used to amplify fragments of genomic or cDNA templates using low stringency polymerase chain reaction conditions. The mixture of primers are designed so that they allow the amplification of gene fragments with related but different sequences. The amplification products can be isolated and used to clone the full length gene, both genomic and cDNA clones. A major advantage of the degenerate PCR approach is that it aids the isolation of members of a specific gene-family of interest.

Identification of G-protein coupled receptor genes that are expressed in inflammatory cells in neutrophil-associated mediated inflammatory disease conditions would provide an important opportunity for the understanding of the inflammatory conditions from which a number of clinically important applications would arise. GPCRs identified may lead to the development of therapeutics (small molecule drugs, antisense molecules, antibody molecules) directly targeted to the gene or protein product of the gene, or may target the biochemical pathway at an upstream or downstream location if the development of such drugs is easier than directly targeting the gene. Polynucleotide sequences comprising the gene and sequence variants thereof may be used to develop a clinical diagnostic test for neutrophil-associated inflammatory disease conditions. Finally, information about the DNA sequences of GPCRs involved in these inflammatory conditions and the amino acid sequences encoded by these genes facilitates large scale production of proteins by recombinant techniques and identification of the tissues and cells naturally producing the proteins. Such sequence information also permits the preparation of antibody substances or other novel binding molecules specifically reactive with the proteins encoded by the GPCR genes that may be used in modulating the natural ligand/antiligand binding reactions in which the proteins are involved.

Accordingly, the present invention provides, in one aspect, the use of (A) a polypeptide comprising the amino acid sequence of SEQ ID NO:2, or a functionally equivalent variant of said amino acid sequence, i.e. a variant thereof which retains a biological or other functional activity thereof, or (B) a polynucleotide, hereinafter alternatively referred to as EX33, comprising a nucleotide sequence encoding the polypeptide (A), or (C) an antibody which is immunoreactive with the polypeptide (A), or (D) an antisense oligonucleotide comprising a nucleotide sequence complementary to that of polynucleotide (B), or (E) a polynucleotide probe comprising at least 15 consecutive nucleotides of (B), in the preparation of a pharmaceutical for the diagnosis or treatment of a neutrophil-associated inflammatory disease.

Terms used herein have the following meanings:

“Isolated” refers to material removed from its original environment.

“Hybridization” or “hybridizes” refers to any process by which a strand of a polynucleotide binds with a complementary strand through base pairing.

“Stringent conditions” refer to experimental conditions which allow up to 20% base pair mismatches, typically two 15 minute washes in 0.1 XSSC (15 mM NaCl, 1.5 mM sodium citrate, pH 7.0) at 65° C.

“Homology” or “homologous” refers to a degree of similarity between nucleotide or amino acid sequences, which may be partial or, when sequences are identical, complete.

“Expression vector” refers to a linear or circular DNA molecule which comprises a segment encoding a polypeptide of interest operably linked to additional segments which provide for its transcription.

“Antisense” refers to selective inhibition of protein synthesis through hybridisation of an oligo- or polynucleotide to its complementary sequence in messenger RNA (mRNA) of the target protein. The antisense concept was first proposed by Zamecnik and Stephenson (Proc. Natl. Acad. Sci. USA 75:280-284; Proc. Natl. Acad. Sci. USA 75:285-288) and has subsequently found broad application both as an experimental tool and as a means of generating putative therapeutic molecules (Alama, A., Pharmacol. Res. 36:171-178; Dean, N. M., Biochem. Soc. Trans. 24:623-629; Bennet, C. F., J. Pharmacol. Exp. Ther. 280:988-1000; Crooke, S. T., Antisense Research and Applications, Springer).

The term “variant” as used herein means, in relation to amino acid sequences, an amino acid sequence that is altered by one or more amino acids. The changes may involve amino acid substitution, deletion or insertion. In relation to nucleotide sequences, the term “variant” as used herein means a nucleotide sequence that is altered by one or more nucleotides; the changes may involve nucleotide substitution, deletion or insertion. A functionally equivalent variant of an amino acid sequence may, for example, be a variant capable of raising an antibody which binds to a polypeptide comprising that amino acid sequence. A preferred functionally equivalent variant of the amino acid sequence SEQ ID NO:2 is one having at least 80%, more preferably at least 90%, and especially more than 95% amino acid sequence identity to SEQ ID NO:2.

By an amino acid sequence having x% identity to a reference sequence such as SEQ ID NO:2, is meant a sequence which is identical to the reference sequence except that it may include up to 100-x amino acid alterations per each 100 amino acids of the reference sequence. For example, in a subject amino acid sequence having at least 80% identity to a reference sequence, up to 20% of the amino acid residues in the reference sequence may be substituted, deleted or inserted with another amino acid residue. Percentage identity between amino acid sequences can be determined conventionally using known computer programs, for example the FASTDB program based on the algorithm of Brutlag et al (Comp.App.Biosci. (1990) 6:237-245).

The polynucleotide (B) may be cDNA, genomic DNA or RNA. In particular embodiments, the polynucleotide (B) is cDNA comprising the nucleotide sequence of SEQ ID NO:1 or a DNA comprising a nucleotide sequence which hybridises to SEQ ID NO:1 under stringent conditions. Nucleotide sequences which satisfy such hybridisation requirements include those resulting from deletions, insertions or substitutions of one or more nucleotides.

In another aspect of the invention, the polynucleotide (B) comprises a consecutive 20 base pair nucleotide portion identical in sequence to a consecutive 20 base pair portion of SEQ ID NO:1. In a further aspect of the invention, the polynucleotide (B) comprises a portion having at least 20, e.g. at least 50 e.g. last least 100, e.g. at least 200, contiguous bases from SEQ ID NO:1. In a yet further aspect of the invention, the polynucleotide (B) comprises a nucleotide sequence encoding at least 10, e.g. at least 50, e.g. at least 100, e.g. at least 200 contiguous amino acids from SEQ ID NO:2.

The polynucleotide (B) may be isolated by first isolating a fragment of it by PCR using degenerate primers that are designed using amino acid sequence motives conserved among members of a family or sub family of GPCRs. The degenerate primers can be used to amplify a fragment from cDNA that have been prepared from RNA isolated from human cells, specifically leukocytes or especially from phagocytes e.g neutrophilic and eosinophilic granulocytes or from genomic DNA. The isolated fragment is then sequenced and full lengths clones are obtained by first isolating overlapping fragments containing the 5′ and 3″ ends of the gene using 5′ and 3′ RACE (rapid amplification of cDNA ends) using gene specific primers designed using that sequence and RNA isolated from human cells, specifically from leukocytes or especially from phagocytes e.g neutrophilic and eosinophilic granulocytes or from genomic DNA and then joining those fragments together by standard methods.

The polynucleotide (B) may also be isolated by first isolating a fragment of it by PCR using degenerate primers that are designed using amino acid sequence motives conserved among members of a family or sub family of GPCRs. The degenerate primers can be used to amplify a fragment from cDNA that have been prepared from RNA isolated from human cells specifically from leukocytes and especially from phagocytes e.g neutrophilic and eosinophilic granulocytes or from genomic DNA. The isolated fragment is then sequenced and full lengths clones are obtained by using this fragment or a part thereof as probe for screening a human cDNA library, preferably a leukocyte or, especially, a granulocyte cDNA library or a human genomic DNA library.

The polynucleotide (B), for example having the sequence SEQ ID NO:1, may be prepared from the nucleotides which it comprises by chemical synthesis, e.g. automated solid phase synthesis using known procedures and apparatus.

The polypeptide (A) may be produced by cloning a polynucleotide sequence as hereinbefore described into an expression vector containing a promoter and other appropriate regulating elements for transcription, transferring into prokaryotic or eukaryotic host cells such as bacterial, plant, insect, yeast, animal or human cells, and culturing the host cells containing the recombinant expression vector under suitable conditions. Techniques for such recombinant expression of polypeptides are well known and are described, for example, in J. Sambrook et al, Molecular Cloning, second edition, Cold Spring Harbor Press, 1990.

In another respect of the invention, the polypeptide (A) comprises a consecutive 10 amino acid portion identical in sequence to a consecutive 10 amino acid portion of SEQ ID NO:2 . In a further aspect, the polypeptide (A) comprises a portion having at least 10, e.g. at least 50, e.g. at least 100, e.g. at least 200, contiguous amino acids from SEQ ID NO:2.

The polypeptide (A) may be expressed as a recombinant fusion protein with one or more heterologous polypeptides, for example to facilitate purification. For example, it may be expressed as a recombinant fusion protein with a heterologous polypeptide such as a polyhistidine containing a cleavage site located between the polynucleotide sequence of the invention and the heterologous polypeptide sequence, so that the polypeptide comprising the amino acid sequence of SEQ ID NO:2 may be cleaved and purified away from the heterologous moiety using well known techniques. The polypeptide (A) may also be synthesised, in whole or in part, from the amino acids which it comprises using well known chemical methods, for example automated solid phase techniques. The polypeptide (A) may be purified by well known standard procedures.

The antibody (C) may be a polyclonal or monoclonal antibody. Such antibodies may be prepared using conventional procedures. Methods for the production of polyclonal antibodies against purified antigen are well established (cf. Cooper and Paterson in Current Protocols in Molecular Biology, Ausubel et al. Eds., John Wiley and Sons Inc., Chapter 11).

Typically, a host animal, such as a rabbit, or a mouse, is immunised with a purified polypeptide of the invention, or immunogenic portion thereof, as antigen and, following an appropriate time interval, the host serum is collected and tested for antibodies specific against the polypeptide. Methods for the production of monoclonal antibodies against purified antigen are well established (cf. Chapter 11, Current Protocols in Molecular Biology, Ausubel et al. Eds., John Wiley and Sons Inc.). For the production of a polyclonal antibody, the serum can be treated with saturated ammonium sulphate or DEAE Sephadex. For the production of a monoclonal antibody, the spleen or lymphocytes of the immunised animal are removed and immortalised or used to produce hybridomas by known methods. Antibodies secreted by the immortalised cells are screened to determine the clones which secrete antibodies of the desired specificity, for example using Western blot analysis. Humanised antibodies can be prepared by conventional procedures.

The antisense oligonucleotide (D) may be DNA, an analogue of DNA such as a phosphorothioate or methylphosphonate analogue of DNA, RNA, an analogue of RNA, or a peptide nucleic acid (PNA). The antisense oligonucleotide may be synthesised by conventional methods, for example using automated solid phase techniques.

The polynucleotide probe (E) comprises at least 15 contiguous nucleotides of a polynucleotide (B) as hereinbefore described or a complement thereof. The probe may be cDNA, genomic DNA or RNA. Usually it is a synthetic oligonucleotide comprising 15 to 50 nucleotides, which can be labelled, e.g. with a fluorophore, to provide a detectable signal. The polynucleotide probe is capable of selectively hybridising under stringent conditions to a polynucleotide fragment having a sequence SEQ ID NO:1. The probe has a sequence such that under such hybridisation conditions it hybridizes only to its cognate sequence. DNA probes as described above are useful in a number of screening applications including Northern and Southern blot analyses, dot blot and slot blot analyses, and fluorescence in situ hybridisation (FISH).

The present invention also includes a pair of oligonucleotides having nucleotide sequences useful as primers for DNA amplification of a fragment of a polynucleotide (B) as hereinbefore described, i.e. of EX33, wherein each primer of said pair is at least 15 nucleotides in length and said pair have sequences such that when used in a polymerase chain reaction (PCR) with either human genomic DNA or a suitable human cDNA target they result in synthesis of a DNA fragment containing all or preferably part of the sequence of EX33. The primer pair is preferably capable of amplifying the coding region of EX33 or portion thereof. Examples of such primer pairs are shown hereinafter as SEQ ID NOs 11-12 and SEQ ID NOs 13-14 respectively.

The role of the polypeptide (A) in neutrophil-associated inflammatory diseases can be determined using a model for the disease, e.g. a lipopolysaccharide-induced lung inflammation model in rat or mouse or models described by Durie et al., Clin. Immunol. Immunopathol. (1994) 73: 11-18; and Williams et al, Proc. Natl. Acad. Sci. USA (1992) 89: 9784-9788.

Polynucleotides, polypeptides, antibodies, antisense oligonucleotides or probes as hereinbefore described, hereinafter alternatively referred to collectively as agents of the invention, may be used in the treatment (prophylactic or symptomatic) or diagnosis of neutrophil-associated inflammatory diseases. For example, a polypeptide (A) of the invention may be used to treat a mammal, particularly a human, deficient in or otherwise in need of that polypeptide; a polynucleotide (B) may be used in gene therapy where it is desired to increase EX33 activity, for instance where a subject has a mutated or missing EX33 gene; or as a diagnostic reagent, e.g. to detect a mutated form of EX33 associated with a neutrophil-associated inflammatory disease; an antisense oligonucleotide (D) may be used to inhibit EX33 activity, where this is desired; an antibody (C) may be used to detect, or determine the level of expression of, EX33 polypeptides, or to inhibit ligand/antiligand binding activities of EX33 polypeptides; and a probe (E) may be used to detect the presence or absence of the EX33 gene, i.e. to detect genetic abnormality. Use of (B), (C) or (E) as a diagnostic reagent may involve detection of under-expression or over-expression of EX33 associated with a particular neutrophil-associated inflammatory disease.

“Gene therapy” refers to an approach to the treatment of human disease based upon the transfer of genetic material into somatic cells of an individual. Gene transfer can be achieved directly in vivo by administartion of gene-bearing viral or non-viral vectors into blood or tissues, or indirectly ex vivo through the introduction of genetic material into cells manipulated in the laboratory followed by delivery of the gene-containing cells back to the individual. By altering the genetic material within a cell, gene therapy may correct underlying disease pathophysiology. Suitable vectors, and procedures, for gene delivery to specific tissues and organ systems in animals are described in Dracopoli, N. C. et al., Current Protocols in Human Genetics. John Wiley and Sons Inc., Chapters 12 and 13 respectively. In relation to polynucleotides (B) as hereinbefore described, gene therapy may involve delivery of a viral or non-viral gene therapy vector containing an expression cassette of the EX33 gene under suitable control elements to the lungs of diseased individuals so that the underlying disease pathophysiology is corrected or ameliorated.

Accordingly, in further aspects, the present invention provides

a pharmaceutical composition for the treatment of a neutrophil-associated inflammatory disease comprising a polypeptide (A), polynucleotide (B), antibody (C) or antisense oligonucleotide (D) as hereinbefore described, optionally together with a pharmaceutically acceptable carrier;

a method of treating a neutrophil-associated inflammatory disease which comprises administering to a subject in need thereof an effective amount of a polypeptide (A), polynucleotide (B), antibody (C) or antisense oligonucleotide (D) as hereinbefore described;

a method of detecting predisposition to a neutrophil-associated inflammatory disease in a subject which comprises incubating a genetic sample from the subject with a polynucleotide probe (E) as hereinbefore defined, under conditions where the probe hybridises to complementary polynucleotide sequence, to produce a first reaction product, and comparing the first reaction product to a control reaction product obtained with a normal genetic sample, where a difference between the first reaction product and the control reaction product indicates a predisposition to a neutrophil-associated inflammatory disease;

a method of diagnosing a neutrophil-associated inflammatory disease in a subject which comprises detecting the presence of a polynucleotide (B) as hereinbefore described, e.g. comprising SEQ ID NO:1, in a cell or tissue from the subject which comprises contacting DNA from the cell or tissue with a polynucleotide probe as hereinbefore defined under conditions where the probe is specifically hybridizable with a polynucleotide (B), and detecting whether hybridization occurs;

a method of detecting an abnormality in the nucleotide sequence of a polynucleotide (B) in a patient which comprises amplifying a target nucleotide sequence in DNA isolated from the patient by a polymerase chain reaction using a pair of primers as hereinbefore described which target the sequence to be amplified and analysing the amplified sequence to determine any polymorphism present therein;

a method of determining whether a subject has or is likely to develop a neutrophil-associated inflammatory disease which comprises determining the level of a polypeptide (A) or polynucleotide (B) as hereinbefore described, or a bioactivity of (A) or the presence of a mutation such as a polymorphism in (B) in a cell or tissue from the subject, and comparing the result with that obtained in a cell or tissue from a healthy subject.

The term “polymorphism” means any sequence difference as compared with the sequence of a polynucleotide of the invention as hereinbefore described.

Hybridisation of a polynucleotide probe (E) with complementary polynucleotide sequence may be detected using in situ (eg. FISH) hybridization, Northern or Southern blot analyses, dot blot or slot blot analyses. The abnormality may also be detected for example by conformation sensitive gel electrophoresis (CSGE) and DNA sequencing as described hereinafter in the Examples. The genetic abnormality may result in a change in the amino acid sequence of the individual's EX33 protein relative to the the amino acid sequence of a normal EX33 protein, or loss of protein. Alternatively, the change may not alter the amino acid sequence but may instead alter expression of the EX33 gene by altering the sequence of controlling elements either at the 5′-, or 3′-end of the gene, or altering the sequence of control elements within intronic regions of the gene. Changes may also affect the way the gene transcript is processed or translated. The invention also includes kits for the detection of an abnormality in the polynucleotide sequence of an individual's EX33 gene. Hybridisation kits for such detection comprise a probe (E) as hereinbefore described, which probe may be modified by incorporation of a detectable, e.g. chemiluminescent or fluorescent, label therein, and may include other reagents such as labelling reagents, i.e. reagents to incorporate a detectable label such as a radioactive isotope, chemiluminescent or fluorescent group into a hybridised product, and buffers. PCR amplification kits comprise primer pairs such as those described above together with a DNA polymerase such as Taq polymerase, and may include additional reagents, such as an amplification buffer and the like. Specific embodiments of the PCR amplification kits can include additional reagents specific for a number of techniques that detect polynucleotide changes, including CSGE and DNA sequencing.

The method of determining whether a subject has or is likely to develop a neutrophil-associated inflammatory disease may comprise determining whether a subject has an abnormal mRNA and/or protein level of EX33, such as by Northern blot analysis, reverse transcription-polymerase chain reaction (RT-PCR), in situ hybridization, immunoprecipitation, Western blot hybridisation or immunohistochemistry. According to the method, cells are obtained from a subject and the EX33 RNA or protein level is determined and compared to the level of EX33 mRNA or protein in a healthy subject. An abnormal level of EX33 is likely to be indicative of an aberrant EX33 activity. In another embodiment, the method comprises measuring at least one activity of EX33, e.g. by measuring intracellular Ca²⁺ as described hereinafter, and comparing the result with that obtained from a healthy subject.

The effectiveness of an agent of the invention in inhibiting or reversing a neutrophil-associated inflammatory disease may be demonstrated in a model of the disease, e.g. a lipopolysaccharide-induced lung inflammation model in rat or mouse or models described by Durie et al., Clin. Immunol. Immunopathol. (1994) 73: 11-18; and Williams et al, Proc. Natl. Acad. Sci. USA (1992) 89: 9784-9788.

The agents of the invention may be administered by any appropriate route, e.g. orally, for example in the form of a tablet or capsule; parenterally, for example intravenously; topically, e.g. in an ointment or cream; transdermally, e.g. in a patch; by inhalation; or intranasally.

Pharmaceutical compositions containing agents of the invention may be prepared using conventional diluents or excipients and techniques known in the galenic art. Thus oral dosage forms may include tablets and capsules, and compositions for inhalation may comprise aerosol or other atomizable formulations or dry powder formulations.

The invention includes (i) an agent (A), (B), (C), (D), or (E) of the invention in inhalable form, e.g. in an aerosol or other atomizable composition or in inhalable particulate, e.g. micronised form, (ii) an inhalable medicament comprising an agent of the invention in inhalable form; (iii) a pharmaceutical product comprising such an agent of the invention in inhalable form in association with an inhalation device; and (iv) an inhalation device containing an agent of the invention in inhalable form.

Dosages of agents of the invention employed in practising the present invention may of course vary depending, for example, on the particular condition to be treated, the effect desired and the mode of administration. In general, suitable daily dosages for administration by inhalation are of the order of 1 μg to 10 mg/kg while for oral administration suitable daily doses are of the order of 0.1 mg to 1000 mg/kg.

A polypeptide (A) as hereinbefore described can be used to identify enhancers (agonists) or inhibitors (antagonists) of its activity, i.e. to identify compounds useful in the treatment of a neutrophil-associated inflammatory disease. Accordingly, the invention also provides a method of identifying a substance suitable for use in treatment of a neutrophil-associated inflammatory disease which modulates the activity of a polypeptide (A) comprising combining a candidate substance with the polypeptide (A) and measuring the effect of the candidate substance on said activity. The activity of a polypeptide (A) may be measured, for example, by measuring intracellular Ca²⁺ or cAMP (cyclic AMP) levels or by a change in shape or by an appropriate reporter gene assay. The invention also includes a method of identifying a substance suitable for use in treatment of a neutrophil-associated inflammatory disease which binds to a polypeptide (A) comprising mixing a candidate substance with a polypeptide (A) and determining whether binding has occurred.

The abovementioned screening methods may be carried out, for example, by preparing cells which express the polypeptide (A) on their surfaces, e.g. insect, mammal or yeast cells and then incubating the resulting cells with the candidate substance to determine the enhancement or inhibition of a functional activity of polypeptide (A) or binding of the candidate substance to the polypeptide (A).

Neutrophil-associated inflammatory diseases to which the present invention is applicable include neutrophil-associated inflammatory or obstructive airways diseases, particularly chronic obstructive pulmonary disease (COPD), including chronic bronchitis and emphysema, and adult (or acute) respiratory distress syndrome (ARDS), rheumatoid arthritis and inflammatory bowel diseases such as Crohn's disease and ulcerative colitis.

The invention is illustrated by the following Examples. Abbreviations used in the Examples have the following meanings:

| | |
|---|---|
| BLAST: | basic local alignment search tool |
| BSA: | bovine serum albumin |
| cAMP: | cyclic adenosine monophosphate |
| DTT: | dithiothreitol |
| EDTA: | ethilene-diamine tetra acetic acid |
| EIA: | enzyme immunoassay |
| EST: | expressed sequence tag |
| FCS: | fetal calf serum |
| GM-CSF: | granulocyte macrophage colony stimulating factor |
| GPCR: | G-protein coupled receptor |
| HEPES: | 4-(2-hydroxyethyl)piperazine-1-ethanesulfonic acid |
| IPTG: | isopropyl-b-D-thiogalactopiranoside |
| IL1: | interleukin1 |
| MOI: | multiplicity of infection |
| PBS: | phosphate buffered saline |
| PEG: | polyethylene glycol |
| PBMC: | peripheral blood mononuclear cells |
| PCR: | polymerase chain reactio |
| PMSF: | phenylmethylsulfonyl fluoride |
| SDS-PAGE: | sodium dodecyl sulfate polyacrylamide gel electrophoresis |
| TNF: | tumor necrosis factor |

### EXAMPLE 1

Blood (200 ml) is collected in tubes containing sodium citrate under sterile conditions from normal donors with no history of respiratory diseases. Neutrophils are purified by well established methods. PBMC are separated from peripheral blood cells by Ficoll Hypaque (pharmacia) centrifugation. The remaining cell population, mainly granulocytes and erythrocytes, are treated with erythrocyte lysis solution (155 mM NH₄Cl, 10 mM KHCO₃, 0.1 mM EDTA, PH 7.3). To determine the purity, granulocytes are stained with Hansel stain (Difco laboratories LTD) and are differentiated by light microscopy at high power magnification. The contamination with eosinophils is found to be less than 2%. For stimulation, neutrophils are resuspended at a concentration of 5 million cells per ml in RPMI-1640 plus 10% FCS. Cells are cultured for 5 hours with or without 50 ng/ml human recombinant GM-CSF (R&D System). For the stimulation of neutrophils via epithelial signals, human primary bronchial epithelial cells (Clonetics) are grown to 70-80% confluency and stimulated simultaneously with TNFα (100 ng/ml) and IL-1β (100 ng/ml) for 48 hrs. Freshly isolated neutrophils are then stimulated by directly adding them to the epithelial cultures or, as a control, to fresh medium. After 5 hrs of incubation neutrophils are carefully washed off the epithelial monolayer and collected for analyses. Total RNA is extracted using TRIZOL Reagent (Gibco/BRL) as described by the manufacturer. One ml of TRIZOL is used for resuspension of every 5 million pelleted neutrophils. mRNA is purified using the MESSAGEMAKER mRNA isolation kit (Gibco/BRL) using conditions recommended by the manufacturer. 300 ng of mRNA is used to synthesize cDNA using the Superscript Choice System (Gibco/BRL). Single stranded and double stranded cDNA are made using conditions provided by the manufacturer.

The resulting cDNA samples are used as templates for the amplification of GPCR gene fragments.

### EXAMPLE 2

This Example describes the isolation of an EX33 cDNA clone.

Degenerate oligonucleotide PCR is performed using primers (SEQ ID NOs 3-6) and conditions as described by Powers et al., J. Exp. Med. (1997) 186:825-835. 100 ng of single stranded cDNA obtained as described in Example 1 is used to seed a 100 μl reaction mixture and subjected to 40 cycles of PCR (95° C. for 1 min, 37° C. for 1 min, and 72° C. for 1 min) using 3 μM of each degenerate oligonucleotide primer:

forward primer: 1:1 mixture of GAY MGI TAY YTI GCI ATH GTI CA and GAY MGI TAY YTI GCI ATH GTC CA

reverse primer: 1:1 mixture of RMR TAI ADI All GGR TTI AIR CA and RMR TAI ADI All GGR TTI ACR CA

in a Perkin-Elmer DNA thermal cycler (Gene Amp PCR System 2400) PCR reaction products are visualized on 1% agarose gels containing 0.5 μg/ml ethidium bromide. Approximately 10 ng of amplified product is ligated to 25 ng pCR2.1 vector (TA Cloning Kit, Invitrogen) and the ligation introduced into 50 μl One Shot™ competent cells (Invitrogen). The libraries are plated onto agar plates containing 50 μg/ml carbenicillin, and 100 mM IPTG and 50 μg/ml X-Gal. Plates are incubated at 37° C. overnight and then briefly at 4° C. to allow blue/white staining to be clearly distinguishable. White colonies are picked into 200 μl of LB-broth containing 50 μg/ml carbenicillin and incubated overnight. Inserts from clones are amplified from cultures using primers corresponding to the T7 and Sp6 sites of the pCR2.1 vector and analysed by electrophoresis on 1.5% agarose gels. Amplified inserts which are of the predicted size (500-550 bp) are analysed by determining their nucleotide sequence on an automated ABI310 sequencer (Perkin-Elmer) using M13 reverse and forward primers. The resulting sequences are analysed in sequence similarity searches using the BLAST algorithm and sequence alignments are done using the GCG software package (Wisconsin Package Version 9.1). A clone called EX33 is identified as containing an insert derived from a GPCR gene. The full length EX33 gene is then isolated by RACE (rapid amplification of cDNA ends). The 5′ and 3′ ends of the EX33 gene are obtained using cDNA isolated from peripheral blood leukocytes and gene-specific primers designed using the sequence of the insert in the EX33 clone. The primer sequences are shown in SEQ ID NO: 7 and NO: 8. RACE is carried out using the 5′ RACE system kit and the 3′ RACE system kit of Life Technologies as suggested by the manufacturer. Amplification products are analysed by determining their nucleotide sequences on both strands on an automated ABI310 sequencer (Perkin-Elmer) using M13 reverse and forward primers as well as gene specific primers. The resulting sequences are analysed and sequence contig is obtained using the GCG software package (Wisconsin Package Version 9.1). The obtained cDNA sequence contig is shown in SEQ ID NO:1.

### EXAMPLE 3

This example describes the analysis of the cellular distribution of EX33 gene expression in various tissues and cell types using Northern blot analysis and RT-PCR.

For Northern blot analyses 20 ug of total RNA isolated from various tissues is separated by electrophoresis in 1% denaturing formaldehyde agarose gels for 4 hours. The RNA is transferred to Hybond N membrane (Amersham) by capillary transfer and UV cross-linked to the filter. Alternatively, premade Multiple Tissue Northern blots (Clontech) are used. To prepare probes, the EX33 containing plasmid (Example 4) is digested with EcoRI and XbaI and the 1.2 kbp insert of the sublcone is isolated from 1% Low Melting Point agarose gels. Probe is labelled by random priming of 25 ng DNA in the presence of [α-³²P] dATP (Amersham). Hybridisations are performed overnight in ExpressHyb hybridisation solution (Clontech) at 65° C. The blots are then washed in 2× SSC, 0.05% SDS for 20 minutes at room temperature, followed by two 20 minute washes in 0.1× SSC, 0.1% SDS at 50° C. Filters are exposed to phosphorimager plates for between 2 hrs and 5 days and visualised by a STORM 840 PhosphorImager (Molecular Dynamics). The same Northern blots are also hybridised with a GAPDH probe to control for loading. It is found that EX33 is expressed in a tissue restricted pattern being abundant for example in bone marrow, lung and peripheral blood but not for example in brain, kidney or liver.

To determine EX33 expression among human peripheral blood leukocytes, the various leukocytes are separated from blood using standard methods. For example, peripheral blood mononuclear cells (PBMC) are separated from granulocytes and erithrocytes by Ficoll Hypaque (Pharmacia) centrifugation as described in Example 1 and T and B cells are isolated from PBMC by MACS (magnetic assisted cell sorter, Miltenyi Biotec). RNA is then isolated from the various blood cell types as described in Example 1. RT-PCR analysis is performed to determine if the expression of EX33 among leukocytes. It is found that EX33 is expressed preferentially among leukocytes being strongly expressed in granulocytes, such as neutrophils and eosinophils but not in T or B lymphocytes.

### EXAMPLE 4

This example describes the analysis of the cellular distribution of EX33 gene expression in the lung by in situ hybridisation and immunohistochemistry.

For the generation of labeled riboprobes a subclone is constructed containing a 1.2 kbp insert between the EcoRI and XbaI sites of the pCR4-TOPO plasmid (Invitrogen). The insert of this subclone is identical to the insert described below in Example 5 and is generated in an identical manner. In this construct using the T3 promoter in the vector transcribes the antisense strand of the insert that can be used as probe in the in situ hybridisation experiments. The T7 promoter in the vector is used to transcribe the sense strand, which is used as a negative control. Serial tissue sections from paraffin-embedded and frozen lung tissue samples are hybridized with radiolabeled cRNA probes that are synthesized from the 1.2 kb insert. Riboprobes are transcribed in vitro in the presence of ³³P-uridine 5′-triphosphate with T3 (antisense) and T7 (sense) RNA polymerases. The probes are then column-purified and then subjected to electrophoresis on a 5% TBE-urea acrylamide gel to confirm size and purity. Tissue sections are digested with Proteinase K and then hybridized with the probes at approximately 8.0×10⁸ dpm/ml at 65° C. for 18 hours. Slides are treated with RNAse A and washed stringently for 2 hours in 0.1× SSC at 65° C. The slides are then coated with Kodak NTB-2 emulsion, exposed for 7 days at 4° C., and developed using Kodak D-19 Developer and Fixer. Slides are stained with hematoxylin and eosin and imaged using a Sony Digital Photo Camera attached to a Nikon microscope. In addition to hybridization with the antisense probe, alternate sections are hybridized with two types of control probes. All tissues are initially screened with a probe for beta-actin mRNA to ensure that RNA has been preserved within the samples. Adjacent serial sections are also hybridized with a sense control riboprobe derived from the same region of the gene as the antisense probe.

The images are evaluated and it is found that the EX33 gene is preferentially expressed in lung alveolar macrophages and in neutrophils.

Expression of the EX33 protein is then studied by immunohistochemistry using standard methods. Experiments are performed on similar tissue sections as for the in situ hybrididsation described above using polyclonal antibodies generated as described in Example 7. Protein expresssion is detected in neutrophils and macrophages confirming the results of the in situ hybridisation experiments.

### EXAMPLE 5

This example relates to the expression of the full length functional EX33 in a mammalian expression system using stable transfection and the use of the transfected cells for the identification of natural ligands or artificial agonists of the EX33 protein.

Construction of the Expression Vector

A unique EcoRI site is incorporated 5′ to the EX33 start codon (ATG) by PCR amplification using the following primer:

5′-CTCAGAATTCATCATGTGGAACAGCTCTGACGC-3′

Another primer is used to introduce a unique XbaI (TCTAGA) site 3′ to the EX33 stop codon (TAG, reverse complement: CTA).

5′-TGGTGTCTAGAGTCACAGTTCTAATGGAGCC-3′

The recombinant amplified product is digested with EcoRI and XbaI restricion enzymes and ligated as a 1213 bp fragment into EcoRI/XbaI digested pcDNA3.1(+) (Invitrogen) mammalian expression vector and transformed into E. coli DH5α cells. Transformants are selected using the ampicillin resistance gene present on pcDNA3.1(+) and recombinant vectors containig the EX33 insert are identified by isolating plasmids from randomly selected colonies and analysing the plasmids by restriction digestion and agarose gel electrophoresis using standard methods.

Stable Expression of EX33 in Mammalian Cells

The recombinant EX33 insert containing plasmid vector is then transfected into CHO-K1 cells. A confluent flask of CHO cells grown in Dulbecco's Modified Eagle's Medium/Ham's f12 (50:50) with 10% FCS and 2 mM glutamine is trypsinized and plated at a dilution of 1:20 into 2 wells of a 6-well plate in 2 ml/well of the same medium. Cells are then incubated for 24 h at 37° C. with 5% CO₂. The next day the transfection mix is prepared. 1 μg plasmid DNA is mixed into 100 μl OptiMEM serum free medium (Life Technologies) for each well. For each well 10 μl Lipofectamine is diluted in 100 μl OptiMEM in a separate tube. The two solutions are mixed and incubated for 15 min at room temperature. During incubation, cells are washed with OptiMEM to remove serum. 0.8 ml/transfection of serum-free OptiMEM is then added to the DNA-liposome transfection mix and 1 ml of that solution is then added to each well. Control cells are treated in an identical manner but omitting plasmid DNA from the transfection mix. Cells are then incubated for 5 h at 37° C. with 5% CO₂ and then the transfection mix is replaced with 2 ml normal growth medium. After 24 h transfectants are selected by washing cells in PBS, trypsinizing and re-plating them into T75 flasks with 1 mg/ml G418 (Life Technologies). Cells are then incubated at 37° C. with 5% CO₂, regularly changing the medium every 2 days until cells in the control flask have died. Cells are then dilution cloned by placing them at a density of one cell per well into individual wells of a 96-well plate and growing them to confluence. After further expansion of the cells individual colonies are screened for the expresssion of EX33 by RT-PCR and by using poly- or monoclonal antibodies raised against the EX33 protein.

Identification of Natural Ligands and Agonists Using Intracellular Calcium Assay

The transfectant cell line stably expressing the EX33 receptor protein and non transfected controls are grown to confluence in T162 flasks, trypsinized and resuspeneded in an appropriate volume, approximately 50 ml of growth medium with no antibiotic. Cells are seeded at 30,000 cells/well in 100 μl/well into 96-well plates that will allow the formation of confluent mono-layers at the time of assay the next day. After 24 h cells are incubated with cytoplasmic calcium indicator Fluo-3-AM (4 mM) in 100 ml cell culture medium containing 20 mM HEPES and 2.5 mM probenecid at 37° C. for 60 min. Cells are washed 4 times with PBS containing 20 mm HEPES and 2.5 mM probenecid and 100 ml of that solution is then added to each well. The test compounds from collections of natural ligands and synthetic compound libraries are added to the cells and the fluorescent signal is read every second for the first 60 seconds and every 5 seconds for the next 30 seconds. Natural or synthetic agonists are identified by comparing the level of signal generated by the same compound in EX33 expressing and control cells.

### EXAMPLE 6

This Example relates to the expression of full length EX33 with a 6 histidine tag after the ATG start codon using the Baculovirus system in Spodoptera frugiperda Sf9 cells, and to the purification of the resulting polypeptide.

Construction of a Recombinant EX33 Baculovirus

A unique EcoRI site is incorporated 5′ to the EX33 start codon (ATG) by PCR amplification using the following primer:

5′-CTCAGAATTCATCATGTGGAACAGCTCTGACGC-3′

Another primer is used to introduce a unique XbaI (TCTAGA) site 3′ to the EX33 stop codon (TAG, reverse complement: CTA).

5′-TGGTGTCTAGAGTCACAGTTCTAATGGAGCC-3′

The recombinant amplified product is digested with EcoRI and XbaI restricion enzymes and ligated as a 1213 bp fragment into EcoRI/XbaI digested pFastbac™HTa baculovirus transfer vector (Life Technologies). In this construct the EX33 gene is expressed as a fusion potein as the EX33 coding region is placed after a 6× His affinity tag followed by a spacer region, a recognition site for TEV protease and an additional 7 amino acid linker region. Expression of the EX33 fusion protein containing the 6× His tag aids affinity purification and the TEV protease cleavage site is used to remove the 6× His tag. The recombinant EX33 sequence is transposed into Bacmid DNA carried by DH10Bac cells (Life Technologies; Bac to Bac Baculovirus expression system). EX33 recombinant Bacmids are isolated from DH10Bac cells and successful transposition is confirmed by PCR analyses.

Transfection of Sf9 Cells with Recombinant EX33 Bacmid DNA and Amplification of Recombinant Baculovirus Stocks

Recombinant EX33 Bacmid DNA is transfected into Sf9 cells using published protocols (Bac to Bac baculovirus expression system manual; Life Technologies). Recominant baculoviruses are harvested from the culture medium after 3-day incubation at 27° C. The cell supernatants are clarified by centrifugation for 5 min at 500× g and kept at 4° C. The recombinant Baculovirus is amplified by infecting Sf9 cells (SF900 SFMII medium; Life Technologies) at a cell density of 1×10⁶ cells/ml and a multiplicity of infection (MOI) of 0.01 for 48 hours. Sf9 cells are then centrifuged at 1000× g for 5 minutes. The supernatants containing high titre virus are stored at 4° C.

Expression of Recombinant EX33 in Sf9 Cells

Sf9 cells, maintained at densities of between 2×10⁵ and 3×10⁶ cells/ml in SF900 SFMII medium; Life Technologies) in either shaker flasks (rotated at 90 RPM) or spinner flasks (stirring at 75 RPM) are infected with the amplified recombinant Baculovirus at a cell density of 1.5×10⁶ at an MOI of 2.0 for 60 hours. Following infection Sf9 cells are centrifuged at 1000× g for 5 minutes, the supernatants poured off and the cell pellets frozen at −80° C.

Crude Lysate Preparation

The cells (1×10⁹) are resuspended in 100 ml lysis buffer (20 mM Hepes pH 7.9, 100 mM NaCl, 5% glycerol, 2 mM E-mercaptoethanol, 0.5 mM imidazole, 0.1% Nonidet P-40, 40 pg/ml AEBSF, 0.5 pg/ml leupeptin, 1 pg/ml aprotinin and 0.7 pg/ml pepstatin A). Cells are incubated on ice for 15 min then centrifuged at 39,000× g for 30 min at 4° C.

Metal Chelate Affinity Chromatography

Metal chelate affinity chromatography is carried out at room temperature with a column attached to a BioCAD chromatography workstation. A 20 ml Poros MC/M (16 mmD×100 mmL) column is charged with Ni²⁺ prior to use and after each injection. To charge with Ni²⁺, the column is washed with 10 column volumes (CV) 50 mM EDTA pH 8, 1 M NaCl followed by 10CV water. The column is charged with 500 ml 0.1 M NiSO4 pH 4.5-5, washed with 10CV water, then any unbound Ni²⁺ removed by washing with 5CV 0.3 M NaCl. All steps are performed with a flow rate of 20 ml/min. The charged MC/M column is equilibrated with 5CV Buffer B (20 mM Hepes pH 7.9, 100 mM NaCl, 5% glycerol, 2 mM E-mercaptoethanol, 1 mM PMSF, 100 mM imidazole) to saturate the sites followed by 10CV Buffer A (as Buffer B except 0.5 mM imidazole). 90-95 ml of the crude lysate is loaded onto the column per run at a flow rate of 20 ml/min. Subsequent steps are carried out with a flow rate of 30 ml/min. Any unbound material is removed by washing with 12 CV buffer A and EX33 eluted by applying a 0-50% Buffer B gradient over 10 CV. Fractions (8 ml) are collected over the gradient. EX33 containing fractions are combined and protease inhibitors added to the final concentrations described for the lysis buffer above. DTT is also added to a final concentration of 1 mM. The combined fractions are dialysed overnight against 4 liters 20 mM Hepes pH 7.9, 1 mM DTT, 0.2 mM PMSF at 4° C. The protein concentartion is determined and if needed, samples are concentrated using a Millipore Ultrafree-15 centrifugation device (MW cut-off 50 kDa) at 4° C. The device is pre-rinsed with water prior to use. The final storage buffer used for long term storage at −80° C. ias 20 mM Hepes pH 7.9, 1 mM DTT, ˜100 mM NaCl, 5% glycerol. Glycerol can be omitted from the sample for storage at 4° C.

### EXAMPLE 7

This example relates to the generation of polyclonal antibodies against the EX33 protein

Immunisation

Rabbits are immunised at 4 subcutaneous sites with 500 μg purified EX33 protein according to the following schedule:

| DAYS | IMMUNISATIONS |
|---|---|
| 0 | 1^{st} immunisation 1:1 in complete Freund's adjuvant |
| 15 | 1^{st} boost 1:1 in incomplete Freund's adjuvant |
| 45 | 2^{nd} boost 1:1 in incomplete Freund's adjuvant |
| 55 | 1^{st} test bleed from the ear artery |
| Every month | Boost 1:1 in incomplete Freund's adjuvant until a good |
| | antibody response is obtained |

Test bleeds (500 μl) are taken and the serum assessed for antibody titre. Serum is collected when a maximum titre is reached. This is done by collecting blood (10 ml) and allowing it to clot for 2 hours at 4° C. The blood is centrifuged at 1000× g for 5 minutes to separate the serum. The serum is removed and stored at −20° C. until assayed.

ELISA Screening

Nunc-Immuno Plate Maxisorp 96 well plates (Nunc, Basle, CH) are used as a solid support and coated with the purified EX33 protein (100 ng/well) o/n at 4° C. The plates are blocked for 3 hours at 37° C. with PBS containing 2% BSA (Sigma) and 0.02% NaN₃ (Sigma). After blocking, plates are incubated overnight at room temperature with plasma in different dilutions of PBS. The presence of polyclonal antibodies is checked with both biotin labelled IgG-antibodies to rabbit (Goat anti-rabbit IgG antiserum, 1:25000 dilution), with an incubation time of 40 min. Alkaline phosphatase conjugated streptavidin (Immununo Research, Dianova, CH) is then added at a dilution of 1:10000. Development of the reaction is carried out by adding phosphate substrate (Sigma, f.c. 1 mg/ml) dissolved in diethanolamine. After 45 min. absorbance is read at 405 nm with a reference of 490 nm with an ELISA plate reader (Biorad).

Purification of the Polyclonal Antibodies

5 ml protein A-agarose is poured into a chromatography column and washed with 6 column volumes of 0.1 M tris (hydroxymethyl) methylamine (Tris) buffer pH 7.5. The rabbit serum containing anti-EX33 antibodies is diluted (½) with Tris buffer and added to the protein A-agarose. Unbound proteins are removed by washing the column with 6 volumes of Tris buffer. The IgG is eluted off the column with three column volumes of 0.1 M glycine buffer pH 3.0 and collected as 1 ml fractions into tubes containing 28 μl of 1 M Tris. The fractions which are positive for protein content are checked for purity by SDS-PAGE under reducing conditions. Two bands at 50 and 25 Kd are visualised corresponding to the heavy and light chains of an immunoglobulin molecule. Fractions containing only immunoglobulin are pooled, re-checked for protein concentration and stored at −20° C.

### EXAMPLE 8

This example relates to the generation of monoclonal antibodies against the EX33 protein

Immunisation

Female Balb/c mice are immunised intraperitoneally (ip) with 100 μg of EX33 protein according to the schedule given below:

| DAYS | IMMUNISATIONS |
|---|---|
| 1 | 1^{st} immunisation 1:1 with complete Freund's adjuvant |
| 14 | 1^{st} boost 1:1 with incomplete Freund's adjuvant |
| 21 | 2^{nd} boost 1:1 with incomplete Freund's adjuvant |
| 28-30 | Three final boosts in PBS |
| 31 | Fusion with mouse myeloma cells |

Serum is assessed for antibody titre by ELISA (Example 7) after the animal is sacrificed for the preparation of spleen cells for fusion. If antibody titre is sufficient, (1/1000 to 1/100,000), the hybridomas are screened, otherwise discarded.

Preparation of Myeloma Cells

Sp^{2/0} murine myeloma cells (ATCC #CRL 1581; maintained in culture medium containing 20 μg/ml 8-azaguanine) are cultivated for one week before fusion in RPMI 1640 (8-azaguanine is not included), 10% (v/v) FCS and 1% penicillin-streptomycin (50 IU/ml and 50 μg/ml, respectively). The cells are harvested by centrifugation (200× g for 5 min) and washed three times in cold RPMI 1640. Approximately 2.5×10⁶ cells are used per 96 well microtitre plate.

Preparation of Spleen Cell Suspension

The mouse is killed by an overdose of anesthetic (Forene), the spleen dissected and pressed through a cell strainer (70 μm mesh cell strainer; Becton & Dickinson, Oxford, UK, Cat. No 2350). The cell suspension is washed three times in RPMI 1640 (as above) and counted: 5.10⁶ cells/96 well plate are necessary.

Fusion of Myeloma Cells and Spleen Cells

The spleen and myeloma cells are mixed (2:1), centrifuged (200× g for 5 min) and the pellet warmed in a 37° C. water bath. Prewarmed polyethylene glycol 4000 (1 ml per 10⁸ cells) is added slowly over one minute, then 20 ml of prewarmed wash medium over two minutes. After centrifugation the pellet is carefully resuspended in selection medium (RPMI 1640, 10% FCS, 1% penicillin-streptomycin, 10% BM condimed H1 (feeder cell replacement from Boebringer Mannheim, Lewes, UK; Cat. No. 1 088 947), 10% HAT-media supplement (hypoxanthine, aminopterin and thymidine to select against unfused myeloma cells; Boehringer Mannheim, Lewes, UK; Cat. No. 644 579) and plated, 200 μl/well of a 96 well microtitre plate.

After five days clusters of hybrid cells can be identified by examining the bottom of the microtitre wells with an inverted microscope. After 10-14 days the culture supernatant is tested for the presence of antibodies by ELISA (Example 7). The positive clones are expanded in a 24 well assay plate and retested.

Cloning of Positive Hybridomas

The expanded clones which are still positive are cloned by limiting dilution. Cells are diluted serially in four dilutions steps in a 96 well microtitre plate; 5, 2, 1 and 0.5 cells/well. HAT-media supplement is replaced with HT-media supplement (Boehringer Mannheim, Lewes, UK; Cat. No. 623 091). After approximately one week the cells are screened by ELISA (Example 7). The cells of those wells containing a single positive clone are expanded.

Production of Monoclonal Antibody Supernatant

The cells are grown in culture flasks in standard medium (RPMI 1640, 10% (v/v) FCS and 1% penicillin-streptomycin) until the hybridomas overgrow and die. The debris is removed by centrifugation and the supernatant containing the antibodies is titred using ELISA (Example 7) before storing under sterile conditions at 4° C., −20° C. or −70° C.

What is claimed is:

1. A method of treating a neutrophil-associated inflammatory disease which comprises administering to a subject in need thereof an effective amount of a pharmaceutical comprising (A) a polypeptide comprising the amino acid sequence of SEQ ID NO:2, or a functionally equivalent variant of said amino acid sequence, or (B) a polynucleotide comprising a nucleotide sequence encoding the polypeptide (A), or (C) an antibody which is immunoreactive with the polypeptide (A), or (D) an antisense oligonucleotide comprising a nucleotide sequence complementary to that of polynucleotide (B).

2. A method according to claim 1 in which the pharmaceutical is a polypeptide (A) comprising a consecutive 10 amino acid portion identical in sequence to a consecutive 10 amino acid portion of SEQ ID NO:2.

3. A method according to claim 1 in which the pharmaceutical is a polynucleotide (B) which is cDNA comprising the nucleotide sequence of SEQ ID NO:1, or a DNA comprising a nucleotide sequence which hybridises to SEQ ID NO:1 under stringent conditions.

4. A method according to claim 3 in which the polynucleotide (B) comprises a consecutive 20 base pair nucleotide portion identical in sequence to a consecutive 20 base pair portion of SEQ ID NO:1.

5. A method according to claim 3, in which the polynucleotide (B) comprises a portion having at least 50 contiguous bases from SEQ ID NO:1.

6. A method according to claim 1 in which the pharmaceutical is an antisense oligonucleotide (D) comprising a nucleotide sequence complementary to that of SEQ ID NO:1.

7. A method according to claim 1, in which the disease is chronic obstructive pulmonary disease, adult respiratory distress syndrome, rheumatoid arthritis or inflammatory bowel disease.

8. A method of detecting a predisposition to developing a neutrophil-associated inflammatory disease in a subject which comprises incubating a genetic sample from the subject with a polynucleotide probe (E) comprising a nucleotide sequence complementary to that of SEQ ID NO: 1 under conditions where the probe hybridises to complementary polynucleotide sequence, to produce a first reaction product, and comparing the first reaction product to a control reaction product obtained with a normal genetic sample, where a difference between the first reaction product and the control reaction product indicates a predisposition to developing a neutrophil-associated inflammatory disease.

9. A method of diagnosing a neutrophil-associated inflammatory disease in a subject comprising detecting the presence of a polynucleotide (B) as specified in claim 1 in a cell or tissue from the subject which comprises contacting DNA from the cell or tissue with a polynucleotide probe comprising at least 15 contiguous nucleotides of a polynucleotide (B) under conditions where the probe is specifically hybridizable with a polynucleotide (B) and detecting whether hybridization occurs.

10. A method according to claim 9, in which the polynucleotide (B) comprises the nucleotide sequence of SEQ ID NO: 1 or a sequence which hybridises to SEQ ID NO: 1 under stringent conditions.

11. A method according to claim 10, in which the polynucleotide (B) comprises a consecutive 20 base pair nucleotide portion identical in sequence to a consecutive 20 base pair portion of SEQ ID NO: 1.

12. A method according to claim 10, in which the polynucleotide (B) comprises a portion having at least 50 contiguous bases from SEQ ID NO: 1.

13. A method of determining whether a subject has, or is likely to develop, a neutrophil-associated inflammatory disease which comprises determining the level of a polypeptide (A) or a polynucleotide (B) as specified in claim 1 in cell or tissue from the subject, or determining a bioactivity of (A) in a cell or tissue from the subject or determining the presence of a mutation in (B) in a cell or tissue from the subject, and comparing the result with that obtained from a healthy subject.

14. A method according to claim 13, in which the polypeptide (A) comprises a consecutive 10 amino acid portion identical in sequence to a consecutive 10 amino acid portion of SEQ ID NO: 2.

15. A method according to claim 13, in which the polynucleotide (B) comprises the nucleotide sequence of SEQ ID NO: 1 or a sequence which hybridises to SEQ ID NO: 1 under stringent conditions.

16. A method according to claim 15, in which (B) comprises a consecutive 20 base pair nucleotide portion identical in sequence to a consecutive 20 base pair portion of SEQ ID NO: 1.

17. A method according to claim 15, in which (B) comprises a portion having at least 50 contiguous bases from SEQ ID NO: 1.

18. A method of identifying a substance suitable for use in treatment of a neutrophil-associated inflammatory disease which modulates an activity of a polypeptide (A) as specified in claim 1 which comprises combining a candidate substance with said polypeptide (A) and measuring the effect of the candidate substance on said activity of (A).

19. A method according to claim 18, in which the disease is chronic obstructive pulmonary disease, adult respiratory distress syndrome, rheumatoid arthritis or inflammatory bowel disease.

20. A method of identifying a substance suitable for use in treatment of a neutrophil-associated inflammatory disease which comprises combining a candidate substance with a polypeptide (A) as specified in claim 1 and determining whether binding has occurred.
